(19) United States
(12) Patent Application Publication
(10) Pub. No.: US 2003/0017948 A1
(43) Pub. Date: Jan. 23, 2003

(54) Method for producing n-substituted 2,4-diamino-5-fluoro benzonitriles and novel intermediates

(76) Inventor: Achim Hupperts, Dusseldorf (DE)

Correspondence Address: BAYER CORPORATION, PATENT DEPARTMENT, 100 BAYER ROAD, PITTSBURGH, PA, 15205, US

(21) Appl. No.: 10/168,487

(22) PCT Filed: Dec. 12, 2000
(86) PCT No.: PCT/EP00/12566

(30) Foreign Application Priority Data
Dec. 24, 1999 (DE) 19962932.3

(51) Int. Cl. A01N 37/34; C07C 253/14

(52) U.S. Cl. 504/310; 558/343

(57) ABSTRACT

The invention relates to a novel process for preparing N-substituted 2,4-diamino-5-fluoro-benzonitriles which are known as intermediates in the preparation of herbicides, to novel N-substituted 4-bromo-6-fluoro-1,3-phenylenediamines, to novel N-substituted 2-bromo-4-fluoro-5-nitro-anilines and to novel N-substituted 2-bromo-4-fluoro-anilines as intermediates for this process, and to processes for their preparation.

The invention relates to a novel process for preparing N-substituted 2,4-diamino-5-fluoro-benzonitriles which are known as intermediates in the preparation of herbicides, to novel N-substituted 4-bromo-6-fluoro-1,3-phenylenediamines, to novel N-substituted 2-bromo-4-fluoro-5-nitro-anilines and to novel N-substituted 2-bromo-4-fluoro-anilines as intermediates for this process, and to processes for their preparations.

It is known that certain N-(5-amino-2-cyano-4-fluoro-phenyl)alkanesulfonamides, such as, for example, N-(5-amino-2-cyano-4-fluoro-phenyl)methanesulfonamide, are obtained when appropriate halogenated benzene derivatives, such as, for example, 1-amino-4-cyano-2,5-difluoro-benzene, are heated with alkanesulfonamides, such as, for example, methanesulfonamide, in the presence of an acid binder, such as, for example, potassium carbonate, and in the presence of a diluent, such as, for example, N-methylpyrrolidone (see EP-A-648 772). However, using this process, the desired products are frequently obtained in unsatisfactory yields.

Furthermore, it is known that certain N-(5-amino-2-cyano-4-fluoro-phenyl)-sulfonamides, such as, for example, N-(5-amino-2-cyano-4-fluoro-phenyl)methane-sulfonamide, are obtained when, in a first step, 2-amino-4,5-difluoro-benzonitrile is reacted with sulfonyl halides, such as, for example, methanesulfonyl chloride, in the presence of an acid binder, such as, for example, triethylamine, and in the presence of a diluent, such as, for example, acetonitrile, and the resulting sulfonylation products are reacted, in a second step, with ammonia in the presence of a diluent, such as, for example, tetrahydrofuran (cf. WO-A-99/05098). In this process, too, the desired products are frequently obtained in unsatisfactory yields.

Furthermore, some N-substituted 4-chloro-6-fluoro-1,3-phenylenediamines are already known from the (patent) literature (cf. WO-A-98/37065). However, the corresponding N-substituted 4-bromo-6-fluoro-1,3-phenylenediamines have hitherto not been disclosed.

It has now been found that N-substituted 2,4-diamino-5-fluoro-benzonitriles of the general formula (I)

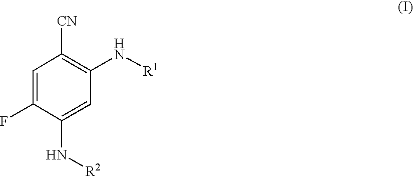

in which

R¹ represents in each case optionally substituted alkylsulfonyl or arylsulfonyl and

R² represents hydrogen or in each case optionally substituted alkoxycarbonyl or aryloxycarbonyl,

are obtained in high yields and in very good quality when, in a first reaction step, 2-bromo-4-fluoro-aniline of the formula (II)

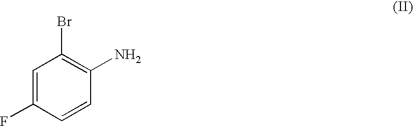

is reacted with an acylating or sulfonylating agent of the general formula (II)

X¹—R¹ (III)

in which

R¹ is as defined above and

X¹ represents halogen,

if appropriate in the presence of one or more reaction auxiliaries and if appropriate in the presence of one or more diluents, at temperatures between −20° C. and +100° C.,

the resulting N-substituted 2-bromo-4-fluoro-anilines of the general formula (IV)

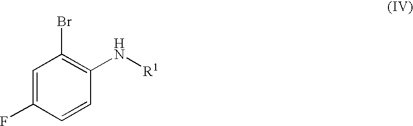

in which

R¹ is as defined above

are, in a second reaction step, reacted with acylating or sulfonylating agents of the general formula (V)

X²—R³ (V)

in which

R³ represents in each case optionally substituted alkylsulfonyl, alkylcarbonyl, alkoxycarbonyl, arylsulfonyl, arylcarbonyl or aryloxycarbonyl and

X² represents halogen,

if appropriate in the presence of one or more reaction auxiliaries and if appropriate in the presence of one or more diluents, at temperatures between −20° C. and 100° C.,

the resulting N-substituted 2-bromo-4-fluoro-anilines of the general formula (VI)

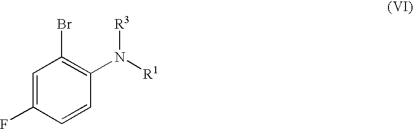

in which

R¹ and R³ are as defined above,

are, in a third reaction step, reacted with nitrating agents, if appropriate in the presence of one or more nitration auxiliaries and/or, if appropriate, in the presence of one or more diluents, at temperatures between −30° C. and +50° C.,

the resulting N-substituted 2-bromo-4-fluoro-5-nitro-anilines of the general formula (VII)

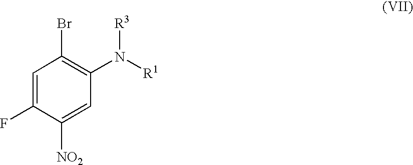

in which

R¹ and R³ are as defined above,

are, in a fourth reaction step, reacted with reducing agents, if appropriate in the presence of one or more reduction auxiliaries and if appropriate in the presence of one or more diluents, at temperatures between 0° C. and 150° C.,

the resulting N-substituted 4-bromo-6-fluoro-1,3-phenylenediamines of the general formula (VIII)

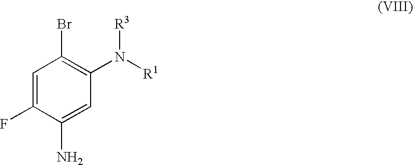

in which

R¹ and R³ are as defined above,

are, if appropriate, in a fifth reaction step, reacted with acylating agents of the general formula (IX)

X³—R³⁻¹ (IX)

in which

R³⁻¹ has the meanings given above for R³ except for hydrogen, and

X³ represents halogen,

if appropriate in the presence of one or more reaction auxiliaries and if appropriate in the presence of one or more diluents, at temperatures between −20° C. and +100° C.,

the N-substituted 4-bromo-6-fluoro-1,3-phenylenediamines, obtained in the fourth or fifth step, of the general formula (X)

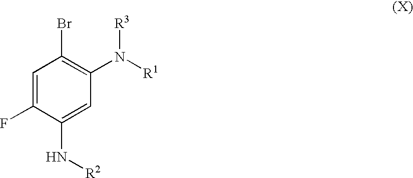

in which

R¹, R² and R³ are as defined above,

are, in a sixth step, reacted with water in the presence of one or more hydrolysis auxiliaries and, if appropriate, in the presence of one or more diluents, at temperatures between 0° C. and 120° C.,

and the resulting N-substituted 4-bromo-6-fluoro-1,3-phenylenediamines of the general formula (XI)

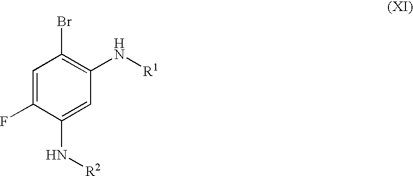

in which

R¹ and R² are as defined above,

are, in a seventh reaction step, reacted with a metal cyanide, if appropriate in the presence of one or more diluents, at temperatures between 50° C. and 200° C.

Surprisingly, the N-substituted 2,4-diamino-5-fluoro-benzonitriles of the general formula (I) prepared by the process according to the invention can be obtained in a relatively simple manner in high yields and in very good quality.

Here, particular mention may be made of the fact that the reduction of the 2-bromo-4-fluoro-5-nitro-anilines of the general formula (VII) to the 4-bromo-6-fluoro-1,3-phenylenediamines of the general formula (VIII) succeeds very well, whereas in general the removal of bromine substituents on arenes is an (undesirable) side reaction frequently observed in the reduction of nitro compounds to the corresponding amino compounds. Since—in contrast to the substitution of a chlorine atom, the exchange of the bromine substituent for a cyano group in the final step proceeds particularly smoothly, the practice of the reaction according to the invention is a very useful addition to the prior art.

The compound 2-bromo-4-fluoro-aniline of the formula (II) to be used as starting material in the first reaction step of the process according to the invention is already known (cf. J. Org. Chem. 28 (1963), 1759-1762).

The acylating or sulfonylating agents of the general formula (III) to be used in the first reaction step are known chemicals for synthesis.

Preferred substituents or preferred ranges of the radicals present in the formulae shown above and below are defined below.

R¹ preferably represents optionally fluorine-, chlorine- or bromine-substituted alkylsulfonyl having 1 to 6 carbon atoms or represents optionally fluorine-, chlorine-, bromine-, C₁-C₄-alkyl- or C₁-C₄-alkoxy-substituted phenylsulfonyl.

R² preferably represents hydrogen, represents optionally fluorine-, chlorine-, or bromine-substituted alkoxycarbonyl having 1 to 6 carbon atoms in the alkyl group, or represents optionally fluorine-, chlorine-, bromine-, C₁-C₄-alkyl- or C₁-C₄-alkoxy-substituted phenoxycarbonyl.

R³ preferably represents in each case optionally fluorine-, chlorine- or bromine-substituted alkylsulfonyl, alkylcarbonyl or alkoxycarbonyl having in each case 1 to 6 carbon atoms in the alkyl groups, or represents in each case optionally fluorine-, chlorine-, bromine-, C₁-C₄-alkyl- or C₁-C₄-alkoxy-substituted phenylsulfonyl, phenylcarbonyl or phenoxycarbonyl.

X¹ preferably represents fluorine, chlorine, bromine or iodine.

X² preferably represents fluorine, chlorine, bromine or iodine.

X³ preferably represents fluorine, chlorine, bromine or iodine.

R¹ particularly preferably represents in each case optionally fluorine- or chlorine-substituted methylsulfonyl, ethylsulfonyl, n- or i-propylsulfonyl, n-, i-, s- or t-butylsulfonyl or represents in each case optionally fluorine-, chlorine-, bromine-, methyl-, ethyl-, n- or i-propyl-, n-, i-, s- or t-butyl-, methoxy-, ethoxy-, n- or i-propoxy-substituted phenylsulfonyl.

R² particularly preferably represents hydrogen, or represents in each case optionally fluorine- or chlorine-substituted methoxycarbonyl, ethoxycarbonyl, n- or i-propoxycarbonyl, n-, i-, s- or t-butoxycarbonyl, or represents optionally fluorine-, chlorine-, bromine-, methyl-, ethyl-, n- or i-propyl-, n-, i-, s- or t-butyl-, methoxy-, ethoxy-, n- or i-propoxy-substituted phenoxycarbonyl.

R³ particularly preferably represents in each case optionally fluorine- or chlorine-substituted methylsulfonyl, ethylsulfonyl, n- or i-propylsulfonyl, n-, i-, s- or t-butylsulfonyl, methylcarbonyl, ethylcarbonyl, n- or i-propylcarbonyl, n-, i-, s- or t-butylcarbonyl, methoxycarbonyl, ethoxycarbonyl, n- or i-propoxycarbonyl, or represents in each case optionally fluorine-, chlorine-, bromine-, methyl-, ethyl-, n- or i-propyl-, n-, i-, s- or t-butyl-, methoxy-, ethoxy-, n- or i-propoxy-substituted phenylsulfonyl, phenylcarbonyl or phenoxycarbonyl.

X¹ particularly preferably represents fluorine, chlorine or bromine.

X² particularly preferably represents fluorine, chlorine or bromine.

X³ particularly preferably represents fluorine, chlorine or bromine.

R¹ very particularly preferably represents in each case optionally fluorine- or chlorine-substituted methylsulfonyl, ethylsulfonyl, n- or i-propylsulfonyl, n- or i-butylsulfonyl.

R² very particularly preferably represents hydrogen or represents methoxycarbonyl, ethoxycarbonyl, n- or i-propoxycarbonyl.

R³ very particularly preferably represents in each case optionally fluorine- or chlorine-substituted methylsulfonyl, ethylsulfonyl, n- or i-propyl-sulfonyl, n- or i-butylsulfonyl, methylcarbonyl, ethylcarbonyl, n- or i-propylcarbonyl, n-, i-, s- or t-butylcarbonyl, or represents methoxycarbonyl, ethoxycarbonyl, n- or i-propoxycarbonyl.

X¹ very particularly preferably represents chlorine.

X² very particularly preferably represents chlorine.

X³ very particularly preferably represents chlorine.

The starting materials of the general formulae (III), (V) and (IX) are known chemicals for synthesis.

The first and the second reaction step of the process according to the invention are preferably carried out in the presence of one or more reaction auxiliaries. Here, suitable reaction auxiliaries are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or -i-propoxide, n-, i-, s- or -t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyl-diisopropylamine, N,N-dimethylcyclohexylamine, dicyclohexylamine, ethyl-dicyclohexyl amine, N,N-dimethylaniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methylpyridine, 4-dimethylaminopyridine, N-methyl -piperidine, 1,4-diazabicyclo[2.2.2]-octane (DABCO), 1,5-diazabicyclo[4.3.0]-non-5-ene (DBN), or 1,8-diazabicyclo[5.4.0]-undec-7-ene (DBU).

The reaction auxiliaries used are preferably basic organic nitrogen compounds, in particular pyridine or triethylamine.

The first and the second reaction step of the process according to the invention are preferably carried out in the presence of one or more diluents. Here, suitable diluents are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropylether, dioxane, tetrahydrofuran or ethylene glycol dimethyl or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; esters such as methyl acetate or ethyl acetate; sulfoxides, such as dimethyl sulfoxide.

The diluents used are preferably halogenated hydrocarbons, in particular methylene chloride, or nitrites, in particular acetonitrile.

When carrying out the first and the second reaction step of the process according to the invention, the reaction temperatures can be varied within a relatively wide range. In general, the reaction steps are carried out at temperatures between −20° C. and +100° C., preferably between −10° C. and +80° C., in particular between 0C and +60° C. The first and the second reaction step of the process according to the invention are generally carried out under atmospheric pressure. However, it is also possible to carry out these reactions under elevated or reduced pressure—in general between 0.1 bar and 10 bar.

For carrying out the first reaction step of the process according to the invention, in general from 1 mol to 5 mol, preferably from 1.0 mol to 2.5 mol, of acylating or sulfonylating agent of the general formula (III) and from 1 to 5 mol, preferably from 1.0 mol to 2.5 mol, of reaction auxiliary are employed per mole of 2-bromo-4-fluoro-aniline of the formula (II).

In a preferred embodiment of the first reaction step, the 2-bromo-4-fluoro-aniline of the formula (II) is initially charged together with a reaction auxiliary in a diluent, and the acylating or sulfonylating agent of the general formula (III) is then slowly metered into this mixture, with stirring (if appropriate with slight cooling). The complete reaction mixture is then (if appropriate at slightly elevated temperature) stirred until the reaction has ended.

For carrying out the second reaction step of the process according to the invention, in general from 1 mol to 3 mol, preferably from 1.0 mol to 1.5 mol, of acylating or sulfonylating agent of the general formula (V) and from 1 to 3 mol, preferably from 1.0 mol to 1.5 mol, of reaction auxiliary are employed per mole of an N-substituted 2-bromo-4-fluoro-aniline of the general formula (IV).

In a preferred embodiment of the second reaction step, the N-substituted 2-bromo-4-fluoro-aniline of the formula (IV) is initially charged together with a reaction auxiliary in a diluent, and the acylating or sulfonylating agent of the general formula (V) is then slowly metered into this mixture, with stirring (if appropriate with slight cooling). The complete reaction mixture is then (if appropriate at slightly elevated temperature) stirred until the reaction has ended.

If, in individual cases, the acylating or sulfonylating agents of the first and the second reaction step are identical, the two steps can be combined in one reaction step, i.e. they are carried out in one step.

Work-up of the products of the first and the second reaction step can be carried out in a customary manner. If the diluent used is virtually immiscible with water, it is possible to wash directly with water. Otherwise (if appropriate after concentration), the mixture is shaken with water and a virtually water-immiscible organic solvent, such as, for example, methylene chloride. From the organic phase, it is then possible, if appropriate after drying and filtration, to obtain the intermediate of the formula (IV) or of the formula (VI) as residue by careful distillative removal of the solvent under reduced pressure. In some cases, the products of the formula (IV) or (VI) can also be obtained in crystalline form by pouring the reaction mixture into aqueous acid (for example hydrochloric acid, if appropriate in a mixture with ice). They can then be isolated by filtration with suction.

Except for the compounds N-(2-bromo-4-fluoro-phenyl)-benzenesulfonamide (cf Izv. Akad. Nauk SSSR, Ser. Khim. (1985), (4), 900-904 - cited in Chem. Abstracts 103:141325) and N-(2-bromo-4-fluoro-phenyl)-1,1,1-trifluoro-methanesulfonamide (cf. J. Org. Chem. (1975), 40 (4), 428-431; US-A-3920444), the N-substituted 2-bromo-4-fluoro-anilines of the general formula (IV) obtainable by the first reaction step of the process according to the invention have hitherto not been disclosed in the literature; except for N-(2-bromo-4-fluoro-phenyl)-benzenesulfonamide and N-(2-bromo-4-fluoro-phenyl)-1,1,1-trifluoro-methanesulfonamide, they form, as novel substances, also part of the subject-matter of the present application.

The N-substituted 2-bromo-4-fluoro-anilines of the general formula (VI) obtainable by the second reaction step of the process according to the invention have hitherto not been disclosed in the literature; as novel substances, they also form part of the subject-matter of the present application.

The third reaction step of the process according to the invention is carried out using a nitrating agent. The nitrating agent used is preferably nitric acid (which, if appropriate, contains up to 30% of water).

The third reaction step of the process according to the invention is, if appropriate, carried out using one or more nitration auxiliaries. Suitable nitration auxiliaries are preferably (virtually anhydrous) acids or acid anhydrides, such as, for example, sulfuric acid, “oleum”, acetic acid or acetic anhydride.

Very particular preference is given to using mixtures of conc. nitric acid and conc. sulfuric acid as mixtures of nitrating agent and nitration auxiliary.

The third reaction step of the process according to the invention is, if appropriate, carried out in the presence of one or more diluents. Suitable diluents are especially inert organic solvents, in particular halogenated alkanes, such as, for example, methylene chloride, chloroform or carbon tetrachloride.

When carrying out the third reaction step of the process according to the invention, the reaction temperatures can be varied within a relatively wide range. In general, the reaction step is carried out at temperatures between −30° C. and +50° C., preferably between −20° C. and +40° C., in particular between −10° C. and +30° C.

The third reaction step of the process according to the invention is generally carried out under atmospheric pressure. However, it is also possible to carry out the third step of the process according to the invention under elevated or reduced pressure—in general between 0.1 bar and 10 bar.

For carrying out the third reaction step of the process according to the invention, in general, from 1 mol to 20 mol, preferably from 2 to 10 mol, of nitrating agent, and from 1 to 50 mol, preferably from 2 to 30 mol, of nitration auxiliary, are used per mole of an N-substituted 2-bromo-4-fluoro-aniline of the general formula (VI).

In a preferred embodiment of the third reaction step, the nitrating agent—preferably together with a nitration auxiliary—is initially charged and the N-substituted 2-bromo-4-fluoro-aniline is metered in—preferably with cooling. The reaction mixture is stirred until the reaction has ended.

Work-up of the products of the third reaction step can be carried out in a customary manner. Preferably, the reaction mixture is poured onto ice, the crystalline product is isolated by filtration with suction and shaken with water and a virtually water-immiscible organic solvent, such as, for example, ethyl acetate, and the organic phase is washed with saturated aqueous sodium bicarbonate solution, dried with sodium sulfate and filtered. Careful removal of the solvent by distillation under reduced pressure gives the N-substituted 2-bromo-4-fluoro-5-nitro-aniline of the formula (VII) as residue.

The N-substituted 2-bromo-4-fluoro-5-nitro-anilines of the general formula (VII) obtainable by the third reaction step of the process according to the invention have hitherto not been disclosed in the literature; as novel substances, they also form part of the subject-matter of the present application.

The fourth reaction step of the process according to the invention is carried out using a reducing agent, if appropriate in the presence of one or more reduction auxiliaries and/or, if appropriate, in the presence of one or more diluents. Here, preference is given to using the reducing agents customary for converting aromatic nitro compounds into the corresponding amino compounds (if appropriate together with suitable reduction auxiliaries and diluents). These preferably include (a) hydrogen in the presence of a catalyst, such as, for example, platinum or palladium (in each case, if appropriate, “poisoned” and on a support, such as, for example, activated carbon or barium sulfate, Raney nickel or Raney cobalt) and in the presence of a diluent, such as, for example, tetrahydrofuran or dioxane, (b) metals or metal salts, such as, for example, tin, tin(II) chloride, iron (powder) in the presence of an acid, such as, for example, hydrochloric acid or acetic acid.

When carrying out the fourth reaction step of the process according to the invention, the reaction temperatures can be varied within a relatively wide range. In general, the reaction step is carried out at temperatures between 0° C. and 150° C., preferably between 10° C. and 120° C., in particular between 20° C. and 100° C.

For carrying out the fourth reaction step of the process according to the invention, in general from 1 mol to 20 mol, preferably from 2 to 10 mol, of reducing agent are employed per mole of an N-substituted 2-bromo-4-fluoro-5-nitro-aniline of the general formula (VII).

In a preferred embodiment of the fourth reaction step, the N-substituted 2-bromo-4-fluoro-5-nitro-aniline is initially charged—preferably together with a reduction auxiliary—and the reducing agent is metered in. The reaction mixture is then stirred until the reaction has ended.

Work-up of the products of the fourth reaction step can be carried out in a customary manner. Preferably, the reaction mixture is filtered and the filtrate is concentrated under reduced pressure. The residue is taken up in a water-immiscible organic solvent such as, for example, ethyl acetate, washed with aqueous sodium bicarbonate solution and dried over sodium sulfate and filtered. Careful removal of the solvent from the filtrate by distillation under reduced pressure gives the N-substituted 4-bromo-6-fluoro-1,3-phenylenediamine of the general formula (VIII) as residue.

The N-substituted 2-bromo-6-fluoro-1,3-phenylenediamines of the general formula (VIII) obtainable by the fourth reaction step of the process according to the invention have hitherto not been disclosed in the literature; as novel substances, they also form part of the subject-matter of the present application.

The fifth reaction step of the process according to the invention is preferably carried out in the presence of one or more reaction auxiliaries. Here, suitable reaction auxiliaries are, in general, the customary inorganic or organic bases or acid acceptors. These preferably include alkali metal or alkaline earth metal acetates, amides, carbonates, bicarbonates, hydrides, hydroxides or alkoxides such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyl diisopropylamine, N,N-dimethyl-cyclohexylamine, dicyclohexylamine, ethyl-dicyclohexylamine, N,N-dimethyl-aniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methyl-pyridine, 4-dimethylamino-pyridine, N-methyl-piperidine, 1,4-diazabicyclo[2.2.2]-octane (DABCO), 1,5-diazabicyclo[4.3.0]-non-5-ene (DBN), or 1,8-diazabicyclo-[5.4.0]-undec-7-ene (DBU).

The reaction auxiliaries used are preferably basic organic nitrogen compounds, in particular pyridine or triethylamine.

The fifth reaction step of the process according to the invention is preferably carried out in the presence of one or more diluents. Here, suitable diluents are especially inert organic solvents. These include, in particular, aliphatic, alicyclic or aromatic, optionally halogenated hydrocarbons such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; esters such as methyl acetate or ethyl acetate; sulfoxides, such as dimethyl sulfoxide.

Preference is given to using aprotic polar solvents from the groups of the ketones, such as, for example, acetone, butanone or methyl isobutyl ketone, of the nitriles, such as, for example, acetonitrile, propionitrile or butyronitrile, or of the amides, such as, for example, N,N-dimethyl-formamide or N,N-dimethyl-acetamide.

When carrying out the fifth reaction step of the process according to the invention, the reaction temperatures can be varied within a relatively wide range. In general, the process step is carried out at temperatures between −20° C. and +100° C., preferably between −10° C. and 80° C., in particular between 0° C. and 60° C.

The fifth reaction step of the process according to the invention is generally carried out under atmospheric pressure. However, it is also possible to carry out the fifth reaction step under elevated or reduced pressure—in general between 0.1 bar and 10 bar.

For carrying out the fifth reaction step of the process according to the invention, in general from 0.9 mol to 1.5 mol, preferably from 0.95 mol to 1.2 mol, of an acylating agent of the general formula (IX) and from 1.0 to 2.0 mol, preferably from 1.1 to 1.5 mol, of a reaction auxiliary are employed per mole of an N-substituted 4-bromo-6-fluoro-1,3-phenylenediamine of the general formula (VIII).

In a preferred embodiment of the fifth reaction step of the process according to the invention, the 4-bromo-6-fluoro-1,3-phenylenediamine of the general formula (VIII) is initially charged with a reaction auxiliary in a diluent, and an acylating agent of the general formula (IX) is metered in, if appropriate with slight cooling. The reaction mixture is stirred until the reaction has ended.

Work-up of the products of the fifth reaction step can be carried out in a customary manner. For example, the reaction mixture can be poured into (ice) water and the product—when it is obtained in crystalline form—can be isolated by filtration with suction. However, it is also possible to shake the reaction mixture with water and a virtually water-immiscible organic solvent, such as, for example, methylene chloride, and to wash the organic phase with water and to dry and filter it. The N-substituted 4-bromo-6-fluoro-1,3-phenylenediamine of the general formula (X) can be obtained as residue by careful removal of the solvent by distillation under reduced pressure.

The N-substituted 4-bromo-6-fluoro-1,3-phenylenediamines of the general formula (X) obtainable by the fifth reaction step of the process according to the invention have hitherto not been disclosed in the literature; as novel substances, they also form part of the subject-matter of the present application.

The sixth reaction step of the process according to the invention is carried out in the presence of one or more hydrolysis auxiliaries. Here, suitable hydrolysis auxiliaries are preferably inorganic bases. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, -n- or -i-propoxide, n-, i-, s- or t-butoxide or potassium methoxide, ethoxide, n- or i-propoxide, n-, i-, s-or t-butoxide.

The hydrolysis auxiliaries used are preferably alkali metal or alkaline earth metal hydrides or hydroxides, in particular sodium hydride, and also sodium hydroxide or potassium hydroxide.

The sixth reaction step of the process according to the invention is, if appropriate, carried out using one or more diluents. Here, suitable diluents are, in addition to water, especially polar organic solvents. These include, in particular, ethers, such as dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; sulfoxides, such as dimethylsulfoxide; alcohols, such as methanol, ethanol, n- or i-propanol, ethylene glycol monomethyl ether, ethylene glycol monoethyl ether, diethylene glycol monomethyl ether, diethylene glycol monoethylether, mixtures thereof with water or pure water.

When carrying out the sixth reaction step of the process according to the invention, the reaction temperatures can be varied within a relatively wide range. In general, the reaction step is carried out at temperatures between −10° C. and +120° C., preferably between 0° C. and 100° C.

In a preferred embodiment of the sixth reaction step of the process according to the invention, an N-substituted 4-bromo-6-fluoro-1,3-phenylenediamine of the general formula (X) is mixed with water and a reaction auxiliary—and, if appropriate, a further diluent—and the reaction mixture is stirred until the reaction has ended.

Work-up of the products of the sixth reaction step can be carried out in a customary manner. For example, the reaction mixture can be poured into (ice) water and, following acidification with a strong acid, such as, for example, hydrochloric acid, the N-substituted 4-bromo-6-fluoro-1,3-phenylenediamine of the general formula (XI) can be isolated as a crystalline product by filtration with suction.

The N-substituted 4-bromo-6-fluoro-1,3-phenylenediamines of the general formula (XI) obtainable by the sixth reaction step of the process according to the invention have hitherto not been disclosed in the literature; as novel substances, they also form part of the subject-matter of the present application.

The seventh reaction step of the process according to the invention is carried out using a metal cyanide. Metal cyanides which may be mentioned here are alkali metal and alkaline earth metal cyanides, such as, for example, sodium cyanide, potassium cyanide, rubidium cyanide, cesium cyanide, magnesium cyanide, calcium cyanide and barium cyanide, but in particular transition metal cyanides, such as copper(I) cyanide. It is also possible to employ mixtures of the metal cyanides mentioned; however, any of these mixtures should comprise copper(I) cyanide.

The seventh reaction step of the process according to the invention is, if appropriate, carried out using one or more diluents. Here, suitable diluents are especially aprotic-polar organic solvents. These include, in particular, ethers, such as dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; esters such as methyl acetate or ethyl acetate; sulfoxides, such as dimethyl sulfoxide.

The diluent used is preferably a high-boiling aprotic-polar organic solvent, such as, for example, N-methyl-pyrrolidone.

When carrying out the seventh reaction step of the process according to the invention, the reaction temperatures can be varied within a relatively wide range. In general, the reaction step is carried out at temperatures between 50° C. and 200° C., preferably between 80° C. and 180° C., in particular between 100° C. and 160° C.

The seventh reaction step of the process according to the invention is generally carried out under atmospheric pressure. However, it is also possible to carry out the seventh step under elevated or reduced pressure—in general between 0.1 bar and 10 bar.

For carrying out the seventh reaction step of the process according to the invention, in general from 1 to 1.5 mol, preferably from 1.1 to 1.3 mol, of a metal cyanide are employed per mole of an N-substituted 4-bromo-6-fluoro-1,3-phenylenediamine of the general formula (XI).

In a preferred embodiment of the seventh reaction step of the process according to the invention, the N-substituted 4-bromo-6-fluoro-1,3-phenylenediamine of the general formula (XI), the metal cyanide and the diluent are mixed at room temperature and stirred at elevated temperature until the reaction has ended.

Work-up of the products of the seventh reaction step can be carried out in a customary manner. For example, the mixture is concentrated under reduced pressure and the residue is stirred with ethyl acetate and hydrochloric-acid-containing aqueous iron (III) chloride solution and filtered through silica gel. The organic phase is then separated off, dried with sodium sulfate and filtered. After removal of the solvent by distillation under reduced pressure, the N-substituted 2,4-diamino-5-fluoro-benzonitrile of the general formula (I) can be obtained as residue.

The compounds of the general formula (I) which can be prepared by the process according to the invention can be used as intermediates for preparing herbicidally active compounds (cf. EP-A-648749, EP-A-648772, WO-A-95/29158).

The intermediates of the general formulae (VIII), (X) and (XI) can likewise be used as precursors for preparing herbicides (cf. EP-A-563384).

The isocyanates of the general formula (XII)

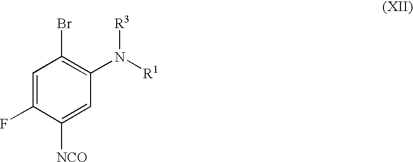

in which

R¹ and R³ are as defined above,

which isocyanates can be prepared from the N-substituted 4-bromo-6-fluoro-1,3-phenylenediamines of the general formula (VIII), can also be used as precursors for herbicides (cf. EP-A-563384).

The isocyanates of the general formula (XII) have hitherto not been disclosed in the literature. As novel substances, they also form part of the subject-matter of the present application.

The novel isocyanates of the general formula (XII) are obtained when N-substituted 4-bromo-6-fluoro-1,3-phenylenediamines of the general formula (VIII)—above—are reacted with phosgene in the presence of a diluent, such as, for example, 1,4-dioxane, at temperatures between 20° C. and 120° C. (cf. the Preparation Examples).

### PREPARATION EXAMPLES

First reaction step:

### Example (IV-1)

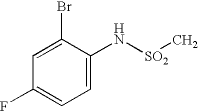

In a 250 ml three-necked flask fitted with internal thermometer, dropping funnel and stirrer, 18.5 g (0.097 mol) of 2-bromo-4-fluoro-aniline are dissolved in 100 ml of methylene chloride and 10 g (0.12 mol) of pyridine and, at 5° C., 12.6 g (0.11 mol) of methane sulfonyl chloride are added. The mixture is stirred at 20° C. for 60 minutes, and 50 ml of water are then added. The phases are separated and the aqueous phase is extracted with 50 ml of methylene chloride. The combined organic phases are washed with 50 ml of water and dried over sodium sulfate. Removal of the solvents using a rotary evaporator gives 21.9 g of pure N-(2-bromo-4-fluoro-phenyl)-methanesulfonamide (84% of theory).

### Example (IV-2)

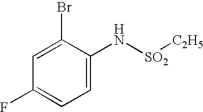

In a 1 l three-necked flask fitted with internal thermometer, dropping funnel and stirrer, 95 g (0.5 mol) of 2-bromo-4-fluoro-aniline are dissolved in 400 ml of methylene chloride and 44 g (0.55 mol) of pyridine and, at from 0° C. to 5° C., 65 g (0.5 mol) of ethanesulfonyl chloride are added. The solution is stirred in an ice bath for 60 minutes and at 20° C. for 3 hours, and 250 ml of water are then added. The phases are separated and the aqueous phase is extracted with 100 ml of methylene chloride. The combined organic phases are washed with 100 ml of water and dried over sodium sulfate. Removal of the solvents using a rotary evaporator gives 135 g of N-(2-bromo-4-fluoro-phenyl)-ethanesulfonamide (91% of theory) which, according to analysis by gas chromatography, is 95.8% pure (melting point: 79-81 ° C.).

First and second reaction step:

### Example (VI-1)

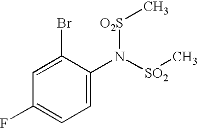

In a 250 ml three-necked flask fitted with internal thermometer, dropping funnel and stirrer, 19.0 g (0.1 mol) of 2-bromo-4-fluoro-aniline are dissolved in 100 ml of acetonitrile and 22.2 g (0.22 mol) of triethylamine and, at from 10° C. —are rel ° C., 25.2 g (0.22 mol) of methanesulfonyl chloride are added. The solution is stirred at 20° C. for 60 minutes and then poured into a mixture of 200 ml of ice-water and 20 ml of conc.hydrochloric acid. The precipitated solid is filtered off with suction and dissolved in 50 ml of methylene chloride, and the organic phase is dried over sodium sulfate. Removal of the solvent using a rotary evaporator gives 30.0 g of N-(2-bromo-4-fluoro-phenyl)-N-methylsulfonyl-methanesulfonamide (87% of theory) which, according to HPLC, is 97.1% pure.

### Example (VI-2)

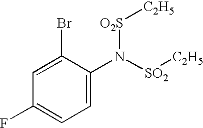

In a 250 ml three-necked flask fitted with internal thermometer, dropping funnel and stirrer, 28.2 g (0.1 mol) of N-(2-bromo-4-fluoro-phenyl)-ethansulfonamide are dissolved in 100 ml of acetonitrile and 11 g (0.11 mol) of triethylamine and, at from 15° C. to 20° C., 13 g (0.1 mol) of ethanesulfonyl chloride are added. The solution is stirred at 20° C. for 60 minutes and then poured into a mixture of 200 ml of ice-water and 20 ml of conc. hydrochloric acid. The precipitated solid is filtered off with suction and dissolved in 50 ml of methylene chloride, and the organic phase is dried over sodium sulfate. Removal of the solvent using a rotary evaporator gives 31.6 g of N-(2-bromo-4-fluoro-phenyl)-N-ethylsulfonyl-ethanesulfonamide (81% of theory) which, according to HPLC, is 95.5% pure (melting point: 114-116° C.).

Second reaction step:

### Example (VI-3)

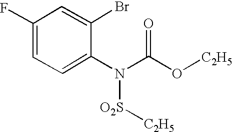

In a 500 ml three-necked flask fitted with internal thermometer, dropping funnel and stirrer, 56.2 g (0.199 mol) of N-(2-bromo-4-fluoro-phenyl)-ethanesulfonamide are dissolved in 200 ml of methylene chloride and 32 g (0.4 mol) of pyridine and, at from 0° C. to 5° C., 23.9 g (0.22 mol) of ethyl chloroformate are added. The solution is stirred at 20° C. for 2 hours, and 100 ml of water are then added. The phases are separated and the aqueous phase is extracted with 50 ml of methylene chloride. The combined organic phases are washed with 100 ml of water and dried over sodium sulfate. Removal of the solvent using a rotary evaporator gives 65.8 g of N-(2-bromo-4-fluoro-phenyl)-N-ethoxycarbonyl-ethanesulfonamide (90.5% of the theory) which, according to GC is 96.9% pure.

Third Reaction Step:

### Example (VII-1)

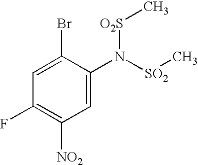

In a 250 ml two-necked flask fitted with internal thermometer and stirrer, 100 ml of conc. sulfuric acid and 35 ml of conc. nitric acid are initially charged and cooled to from 0° C. to 5° C. 24.3 g (0.07 mol) of N-(2-bromo-4-fluoro-phenyl)-N-methyl-sulfonyl-methanesulfonamide are added to this mixture. The mixture is allowed to warm to 20° C. and stirred for another hour, and the reaction solution is then put onto 300 g of ice. The precipitated solid is filtered off with suction and taken up in 100 ml of ethyl acetate. The organic phase is washed with 50 ml of water and then with 50 ml of saturated sodium bicarbonate solution and dried over sodium sulfate. Removal of the solvent using a rotary evaporator gives 26.5 g of pure N-(2-bromo-4-fluoro-5-nitro-phenyl)-N-methylsulfonyl-methanesulfonamide (97% of theory).

### Example (VII-2)

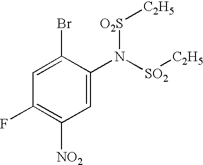

In a 250 ml two-necked flask fitted with internal thermometer and stirrer, 100 ml of conc. sulfuric acid and 35 ml of conc. nitric acid are initially charged and cooled to from 0° C. to 5° C. 29.9 g (0.08 mol) of N-(2-bromo-4-fluoro-phenyl)-N-ethylsulfonyl-ethansulfonamide are then added to this mixture. The mixture is allowed to warm to 20° C. and stirred for another hour, and the reaction solution is then poured onto 300 g of ice. The precipitated solid is filtered off with suction and taken up in 100 ml of ethyl acetate. The organic phase is washed with 50 ml of water and then with 50 ml of saturated sodium bicarbonate solution and dried over sodium sulfate. Removal of the solvent using a rotary evaporator gives 29.1 g of N-(2-bromo-4-fluoro-5-nitro-phenyl)-N-ethylsulfonyl-ethanesulfonamide (81.5% of theory) which, according to HPLC, is 93.9% pure (melting point: 144-146° C.).

### Example (VII-3)

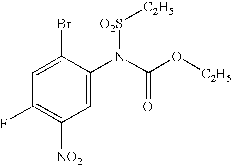

In a 500 ml two-necked flask fitted with internal thermometer and stirrer, 270 ml of conc. sulfuric acid and 10 ml of conc. nitric acid are initially charged and cooled to from 0° C. to 5° C. 90.0 g (0.25 mol) of N-(2-bromo-4-fluoro-phenyl)-N-ethoxy-carbonyl-ethanesulfonamide are added to this mixture. The mixture is allowed to warm to 15° C. and stirred for another 2 hours, and the reaction solution is then poured onto 1 kg of ice. The precipitated solid is filtered off with suction and taken up in 300 ml of ethyl acetate. The organic phase is washed with 100 ml of water and then with 100 ml of saturated sodium bicarbonate solution and dried over sodium sulfate. Removal of the solvent using a rotary evaporator gives 92.3 g of N-(2-bromo-4-fluoro-5-nitro-phenyl)-N-ethoxycarbonyl-ethanesulfonamide (88% of theory) which, according to HPLC is 95.7% pure.

Fourth Reaction Step:

### Example (VIII-1)

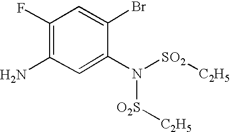

In a 250 ml three-necked flask fitted with reflux condenser, internal thermometer and stirrer, 8.4 g (0.02 mol) of N-(2-bromo-4-fluoro-5-nitro-phenyl)-N-ethylsulfonyl-ethanesulfonamide are dissolved with heating in 100 ml of acetic acid, and 6.7 g (0.12 mol) of iron powder are added at 35° C. The mixture is then heated carefully until the exothermic reaction sets in (about 40° C.), and the temperature is then maintained at about 40° C. by slight cooling using a water bath. After the reaction has ended, the mixture is stirred for another hour and then filtered. The solids are separated off and washed with 20 ml of acetic acid, and the filtrate is concentrated using a rotary evaporator. The residue is taken up in 100 ml of ethyl acetate, washed with 50 ml of water and then with 50 ml of saturated sodium bicarbonate solution and dried over sodium sulfate. Removal of the solvent using a rotary evaporator gives 2.9 g of N-(2-bromo-4-fluoro-5-amino-phenyl)-N-ethylsulfonyl-ethanesulfonamide (34% of theory) which, according to HPLC, is 91.0% pure.

### Example (VIII-2)

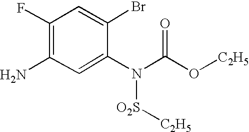

In a 1 l three-necked flask fitted with reflux condenser, internal thermometer and stirrer, 40.0 g (0.1 mol) of N-(2-bromo-4-fluoro-5-nitro-phenyl)-N-ethoxycarbonyl-ethanesulfonamide are dissolved with heating in 400 ml of acetic acid, and 33.0 g (0.6 mol) of iron powder are added at 20° C. The mixture is heated carefully until the exothermic reaction sets in (about 50° C.), and the temperature is then maintained at about 50° C. by slight cooling using a water bath. After the reaction has ended, the mixture is stirred for another hour and then filtered. The solids are separated off and washed with 50 ml of acetic acid, and the filtrate is concentrated using a rotary evaporator. The residue is taken up in 100 ml of ethyl acetate and 100 ml of water, the aqueous phase is neutralized (pH=7-8), and the phases are separated. The org. phase is washed with 100 ml of water and then with 100 ml of saturated sodium bicarbonate solution and dried over sodium sulfate. Removal of the solvent using a rotary evaporator gives 34.3 g of pure N-(2-bromo-4-fluoro-5-amino-phenyl)-N-ethoxycarbonyl-ethanesulfonamide (93% of theory).

Fifth Reaction Step:

### Example (X-1)

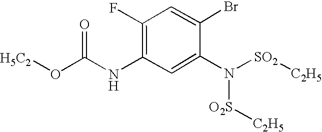

In a 100 ml three-necked flask fitted with internal thermometer, dropping funnel and stirrer, 2.8 g (0.0072 mol) of N-(2-bromo-4-fluoro-5-amino-phenyl)-N-ethylsulfonyl-ethanesulfonamide are dissolved in 50 ml of acetonitrile and 0.8 g (0.01 mol) of pyridine, and 0.9 g (0.008 mol) of ethyl chloroformate are added at 5-10° C. The solution is stirred at 20° C. for 60 minutes and then poured into 100 ml of ice-water. The precipitated solid is filtered off with suction, washed with water and dried. This gives 3.3 g of N-(2-bromo-4-fluoro-5-ethoxycarbonylamino-phenyl)-N-ethylsulfonyl-ethanesulfonamide (99% of theory) as a light-brown solid which, according to GC, is 90.6% pure.

### Example (X-2)

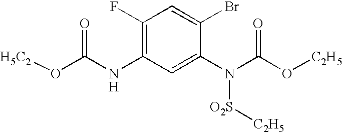

In a 250 ml three-necked flask fitted with internal thermometer, dropping funnel and stirrer, 16.6 g (0.045 mol) of N-(2-bromo-4-fluoro-5-amino-phenyl)-N-ethoxy-carbonyl-ethanesulfonamide are dissolved in 120 ml of chloroform and 4.4 g (0.055 mol) of pyridine and, at from 5° C. to 10° C., 4.9 g (0.045 mol) of ethyl chloroformate are added. The solution is stirred at about 20° C. for 1 h, and 100 ml of water are then added. The phases are separated and the aqueous phase is extracted with 50 ml of methylene chloride. The combined organic phases are washed with 100 ml of water and dried over sodium sulfate. Removal of the solvent using a rotary evaporator gives 17.4 g of N-(2-bromo-4-fluoro-5-ethoxycarbonylamino-phenyl)-N-ethoxycarbonyl-ethanesulfonamide (87% of theory) which, according to HPLC, is 99.9% pure (melting point: 142-143° C.).

### Example (X-3)

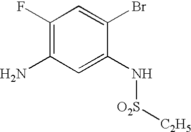

In a 100 ml three-necked flask fitted with internal thermometer, dropping funnel and stirrer, 6.24 g (0,021 mol) of N-(2-bromo-4-fluoro-5-amino-phenyl)-ethane-sulfonamide are dissolved in 50 ml of methylene chloride and 2.0 g (0,025 mol) of pyridine and, at from 15° C. to 20° C., 2.7 g (0.025 mol) of ethyl chloroformate are added. The solution is stirred at 20° C. for 60 minutes, and 100 ml of water are then added. The phases are separated and the aqueous phase is extracted with 50 ml of methylene chloride. The combined organic phases are washed with 100 ml of water and dried over sodium sulfate. Removal of the solvent using a rotary evaporator gives 7.25 g of N-(2-bromo-4-fluoro-5-ethoxycarbonylamino-phenyl)-ethanesulfonamide (91% of theory) which, according to HPLC, is 97.2% pure.

Sixth Reaction Step:

### Example (XI-1)

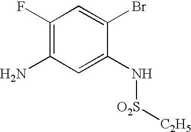

In a 500 ml three-necked flask fitted with internal thermometer and stirrer, 18.5 g (0.05 mol) of N-(2-bromo-4-fluoro-5-amino-phenyl)-N-ethoxycarbonyl-ethane-sulfonamide are dissolved in 100 ml of water and 20 ml of conc. aqueous sodium hydroxide solution and heated at from 60° C. to 70° C. for 4 h. The mixture is allowed to cool to room temperature and acidified carefully with 2N hydrochloric acid (pH 1) and the precipitated solid is filtered off with suction. The solid is washed with water and dried in a dessicator, giving 13.7 g of pure N-(2-bromo-4-fluoro-5-amino-phenyl)-ethanesulfonamide (92% of theory).

### Example (XI-2)

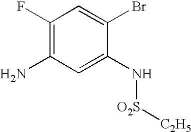

In a 50 ml three-necked flask fitted with internal thermometer and stirrer, 0.2 g (0.005 mol) of sodium hydride (60% pure) are initially charged in 20 ml of N,N-di-methyl-formamide and, with cooling (from 0° C. to 5° C.), 2.15 g (0.0046 mol) of N-(2-bromo-4-fluoro-5-ethoxycarbonylamino-phenyl)-N-ethylsulfonyl-ethanesulfonamide are added. The solution is stirred in an ice bath for 60 minutes and then added to a mixture of 10 ml of conc. hydrochloric acid and 50 ml of ice-water. The precipitated solid is filtered off with suction, washed with water and dried. This gives 1.4 g of N-(2-bromo-4-fluoro-5-ethoxycarbonylamino-phenyl)-ethanesulfonamide (82% of theory) which, according to HPLC, is 78.6% pure.

Seventh Reaction Step:

### Example (I-1)

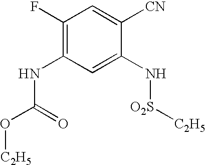

In a 2 l three-necked flask fitted with internal thermometer, reflux condenser and stirrer, 383.8 g (1.00 mol) of N-(2-bromo-4-fluoro-5-ethoxycarbonylamino-phenyl)-ethanesulfonamide and 107.5 g (1.20 mol) of copper(I) cyanide are dissolved in 1 liter of dry N-methyl-pyrrolidone and heated at 140° C. for 6 hours. The mixture is allowed to cool to room temperature, and 800 ml of N-methyl-pyrrolidone are distilled off under oil pump vacuum. 1 liter of ethyl acetate and a solution of iron(III) chloride in hydrochloric acid are added to the residue. The two-phase system is filtered through silica gel and the phases are separated. The organic phase is dried over sodium sulfate and concentrated using a rotary evaporator. The tacky residue is washed by stirring with petroleum ether, filtered off with suction and dried. This gives 275.9 g of N-(2-cyano-4-fluoro-5-ethoxycarbonylamino-phenyl)-ethane-sulfonamide (75% of theory) which, according to HPLC, is 86.0% pure.

### Example (I-2)

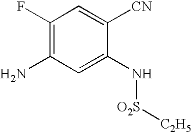

In a 50 ml two-necked flask fitted with internal thermometer, reflux condenser and stirrer, 1.49 g (0.005 mol) of N-(2-bromo-4-fluoro-5-amino-phenyl)-ethane-sulfonamide and 0.49 g (0.0055 mol) of copper(I) cyanide are dissolved in 10 ml of dry N-methyl-pyrrolidone and heated at 130° C. for 6 hours. The mixture is allowed to cool to room temperature and the N-methyl-pyrrolidone is distilled off under oil pump vacuum. According to HPLC, the crude product comprises 76% of N-(2-cyano-4-fluoro-5-amino-phenyl)-ethanesulfonamide and 8.5% of starting material.

Isocyanates of the formula (XII):

### Example (XII-1)

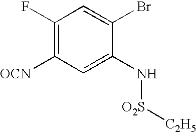

In a 1 l three-necked flask fitted with internal thermometer, gas inlet tube and stirrer, 50.0 g (0.165 mol) of N-(2-bromo-4-fluoro-5-amino-phenyl)-ethanesulfonamide are dissolved in 490 ml of acetonitrile and, at from 15° C. to 20° C., 7.3 g (0.2 mol) of hydrogen chloride gas are added, which results in a white solid precipitating out. At from 20° C. to 60° C. and with stirring, 69.3 g (0.70 mol) of phosgene are then added to the suspension, which is stirred at 60° C. for another 150 minutes. The mixture is allowed to stand overnight, and excess phosgene is distilled off. Removal of the solvent using a rotary evaporator gives 53.9 g of 2-fluoro-4-bromo-5-ethylsulfonyl-amino-phenylisocyanate (97 % of theory), which according to HPLC, is 96.2% pure.

### Example (XII-2)

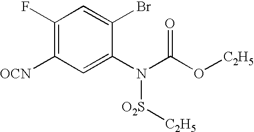

In a 500 ml four-necked flask fitted with internal thermometer, gas inlet tube, dropping funnel and stirrer, 8 g (0.080 mol) of phosgene are dissolved in 220 ml of 1,4-dioxane and, at from 15° C. to 20° C., 5.0 g (0.0135 mol) of N-(2-bromo-4-fluoro-5-amino-phenyl)-N-ethoxycarbonyl-ethanesulfonamide, dissolved in 70 ml of 1,4-dioxane, are added (1 h). The solution is then heated at from 70° C. to 80° C., and a further 35.4 g (0.35 mol) of phosgene are introduced. After about 3 hours, the mixture is allowed to cool to room temperature and excess phosgene is flushed out overnight using nitrogen. Removal of the solvent using a rotary evaporator gives 6.24 g of 2-fluoro-4-bromo-5-N-ethylsulfonyl-N-ethoxycarbonylamino-phenyliso-cyanate (86 % of theory) which, according to HPLC, is 74% pure.

1. Process for preparing N-substituted 2,4-diamino-5-fluoro-benzonitriles of the general formula (I) 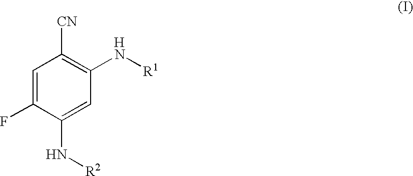
in which
R¹ represents in each case optionally substituted alkylsulfonyl or arylsulfonyl and
R² represents hydrogen or in each case optionally substituted alkoxycarbonyl or aryloxycarbonyl,
characterized in that, in a first reaction steps 2-bromo-4-fluoro-aniline of the formula (II) 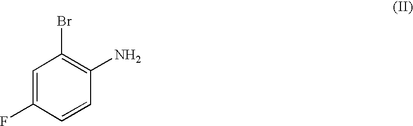
is reacted with an acylating or sulfonylating agent of the general formula (II)
X¹—R¹ (III)
in which
R¹ is as defined above and
X¹ represents halogen,
if appropriate in the presence of one or more reaction auxiliaries and if appropriate in the presence of one or more diluents, at temperatures between −20° C. and +100° C.,
the resulting N-substituted 2-bromo-4-fluoro-anilines of the general formula (IV) 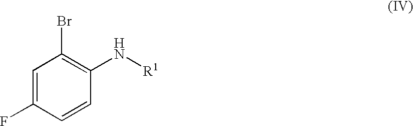
in which
R¹ is as defined above
are, in a second reaction step, reacted with acylating or sulfonylating agents of the general formula (V)
X²—R³ (V)
in which
R³ represents in each case optionally substituted alkylsulfonyl, alkylcarbonyl, alkoxycarbonyl, arylsulfonyl, arylcarbonyl or aryloxy-carbonyl and
X² represents halogen,
if appropriate in the presence of one or more reaction auxiliaries and if appropriate in the presence of one or more diluents, at temperatures between −20° C. and 100° C.,
the resulting N-substituted 2-bromo-4-fluoro-anilines of the general formula (VI) 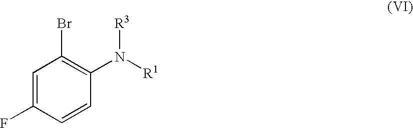
in which
R¹ and R³ are as defined above,
are, in a third reaction step, reacted with nitrating agents, if appropriate in the presence of one or more nitration auxiliaries and/or, if appropriate, in the presence of one or more diluents, at temperatures between −30° C. and +50° C.,
the resulting N-substituted 2-bromo-4-fluoro-5-nitro-anilines of the general formula (VII) 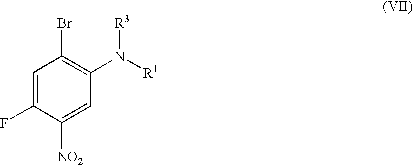
in which
R¹ and R³ are as defined above,
are, in a fourth reaction step, reacted with reducing agents, if appropriate in the presence of one or more reduction auxiliaries and if appropriate in the presence of one or more diluents, at temperatures between 0° C. and 150° C.,
the resulting N-substituted 4-bromo-6-fluoro-1,3-phenylenediamines of the general formula (VIII) 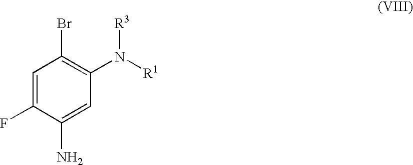
in which
R¹ and R³ are as defined above,
are, if appropriate, in a fifth reaction step, reacted with acylating agents of the general formula (IX)
X³—R³⁻¹ (IX)
in which
R³⁻¹ has the meanings given above for R³ except for hydrogen, and
X³ represents halogen,
if appropriate in the presence of one or more reaction auxiliaries and if appropriate in the presence of one or more diluents, at temperatures between −20° C. and +100° C.,
the N-substituted 4-bromo-6-fluoro-1,3-phenylenediamines, obtained in the fourth or fifth step, of the general formula (X) 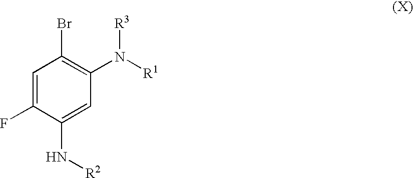
in which
R¹ , R² and R³ are as defined above,
are, in a sixth step, reacted with water in the presence of one or more hydrolysis auxiliaries and, if appropriate, in the presence of one or more diluents, at temperatures between 0° C. and 120° C.,
and the resulting N-substituted 4-bromo-6-fluoro-1,3-phenylenediamines of the general formula (XI) 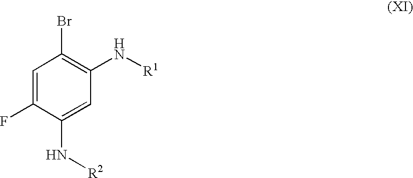
in which
R¹ and R² are as defined above,
are, in a seventh reaction step, reacted with a metal cyanide, if appropriate in the presence of one or more diluents, at temperatures between 50° C. and 200° C.

2. A process as claimed in claim 1, characterized in that
R¹ represents optionally fluorine-, chlorine- or bromine-substituted alkyl-sulfonyl having 1 to 6 carbon atoms or represents optionally fluorine-, chlorine-, bromine-, C₁-C₄-alkyl- or C₁-C₄-alkoxy-substituted phenylsulfonyl,
R² represents hydrogen, represents optionally fluorine-, chlorine-, or bromine-substituted alkoxycarbonyl having 1 to 6 carbon atoms in the alkyl group, or represents optionally fluorine-, chlorine-, bromine-, C₁-C₄-alkyl- or C₁-C₄-alkoxy-substituted phenoxycarbonyl,
R³ represents in each case optionally fluorine-, chlorine- or bromine-substituted alkylsulfonyl, alkylcarbonyl or alkoxycarbonyl having in each case 1 to 6 carbon atoms in the alkyl groups, or represents in each case optionally fluorine-, chlorine-, bromine-, C₁-C₄-alkyl- or C₁-C₄-alkoxy-substituted phenylsulfonyl, phenylcarbonyl or phenoxycarbonyl,
X¹ represents fluorine, chlorine, bromine or iodine,
X² represents fluorine, chlorine, bromine or iodine, and
X³ represents fluorine, chlorine, bromine or iodine.

3. A process as claimed in claim 1, characterized in that
R¹ represents in each case optionally fluorine- or chlorine-substituted methylsulfonyl, ethylsulfonyl, n- or i-propylsulfonyl, n-, i-, s- or t-butylsulfonyl or represents in each case optionally fluorine-, chlorine-, bromine-, methyl-, ethyl-, n-or i-propyl-, n-, i-, s- or t-butyl-, methoxy-, ethoxy-, n- or i-propoxy-substituted phenylsulfonyl,
R² represents hydrogen, or represents in each case optionally fluorine- or chlorine-substituted methoxycarbonyl, ethoxycarbonyl, n- or i-propoxycarbonyl, n-, i-, s- or t-butoxycarbonyl, or represents optionally fluorine-, chlorine-, bromine-, methyl-, ethyl-, n- or i-propyl-, n-, i-, s- or t-butyl-, methoxy-, ethoxy-, n- or i-propoxy-substituted phenoxycarbonyl,
R³ represents in each case optionally fluorine- or chlorine-substituted methylsulfonyl, ethylsulfonyl, n- or i-propylsulfonyl, n-, i-, s- or t-butylsulfonyl, methylcarbonyl, ethylcarbonyl, n- or i-propylcarbonyl, n-, i-, s- or t-butylcarbonyl, methoxycarbonyl, ethoxycarbonyl, n- or i-propoxycarbonyl, or represents in each case optionally fluorine-, chlorine-, bromine-, methyl-, ethyl-, n- or i-propyl-, n-, i-, s- or t-butyl-, methoxy-, ethoxy-, n- or i-propoxy-substituted phenylsulfonyl, phenylcarbonyl or phenoxycarbonyl,
X¹ represents fluorine, chlorine or bromine,
X² represents fluorine, chlorine or bromine, and
X³ represents fluorine, chlorine or bromine.

4. A process as claimed in claim 1, characterized in that
R¹ represents in each case optionally fluorine- or chlorine-substituted methylsulfonyl, ethylsulfonyl, n- or i-propylsulfonyl, n- or i-butylsulfonyl,
R² represents hydrogen or represents methoxycarbonyl, ethoxycarbonyl, n- or i-propoxycarbonyl,
R³ represents in each case optionally fluorine- or chlorine-substituted methylsulfonyl, ethylsulfonyl, n- or i-propylsulfonyl, n- or i-butyl-sulfonyl, methylcarbonyl, ethylcarbonyl, n- or i-propylcarbonyl, n-, i-, s- or t-butylcarbonyl, or represents methoxycarbonyl, ethoxycarbonyl, n- or i-propoxycarbonyl,
X¹ represents chlorine,
X² represents chlorine, and
X³ represents chlorine.

5. An N-substituted 2-bromo-4-fluoro-aniline of the general formula (IV) 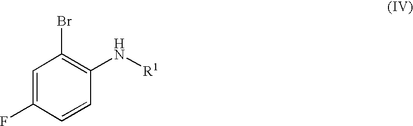
in which
R¹ is as defined in any of claims 1 to 4,
except for the compounds N-(2-bromo-4-fluoro-phenyl)-benzenesulfonamide and N-(2-bromo-4-fluoro-phenyl)-1,1,1-trifluoromethanesulfonamide.

6. An N-substituted 2-bromo-4-fluoro-aniline of the general formula 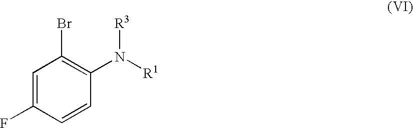
in which
R¹ and R³ are as defined in any of claims 1 to 4.

7. An N-substituted 2-bromo-4-fluoro-5-nitro-aniline of the general formula (VII) 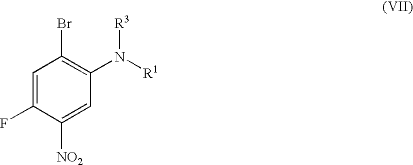
in which
R¹ and R³ are as defined in any of claims 1 to 4.

8. An N-substituted 4-bromo-6-fluoro-1,3-phenylendiamine of the general formula (VIII) 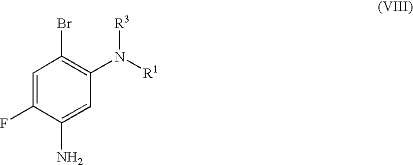
in which
R¹ and R³ are as defined in any of claims 1 to 4.

9. An N-substituted 4-bromo-6-fluoro- 1,3-phenylenediamine of the general formula (XI) 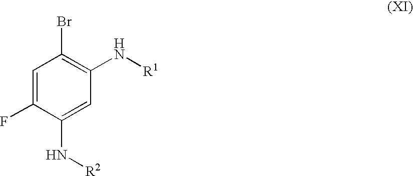
in which
R¹ and R² are as defined in any of claims 1 to 4.

10. A compound of the general formula (XII) 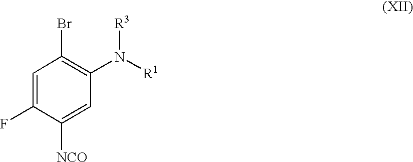
in which
R¹ and R³ are as defined in any of claims 1 to 4.
